(19) United States
(12) Patent Application Publication
(10) Pub. No.: US 2016/0296199 A1
(43) Pub. Date: Oct. 13, 2016

(54) ACOUSTICALLY TRANSPARENT ANTIMICROBIAL SURFACES

(71) Applicant: NITTO DENKO CORPORATION, Ibaraki-shi, Osaka (JP)

(72) Inventors: Rajesh Mukherjee, Oceanside, CA (US); Guang Pan, Oceanside, CA (US); Tao Gu, Oceanside, CA (US); Jiadong Zhou, Oceanside, CA (US)

(21) Appl. No.: 15/035,740

(22) PCT Filed: Nov. 12, 2014
(86) PCT No.: PCT/US2014/065296

§ 371 Date: May 10, 2016

Related U.S. Application Data
(60) Provisional application No. 61/903,256, filed on Nov. 12, 2013.

(51) Int. Cl. A61B 7/02; C23C 14/34; C23C 14/20; A61B 46/10; A61B 8/00

(52) U.S. Cl. CPC A61B 7/02; A61B 46/10; A61B 8/4422; C23C 14/205; C23C 14/34; A61B 2562/12; B05D 1/005

(57) ABSTRACT

Described herein are medical elements for reducing intra-patient microbial contamination. The elements include an acoustically transmissive matrix and an antimicrobial element.

### BACKGROUND

1. Field

Some embodiments are related to stethoscopes and to accessories for stethoscopes.

2. Description of the Related Art

Because a doctor will use a stethoscope on different patients, it has become commonplace for doctor's assistants to place a cloth boot over the metal end of the stethoscope that contacts the patient. This cloth boot is disposable and is replaced for each patient. It has also been recognized that the plastic tubing of the stethoscope may also come into contact with patients and, therefore, various solutions have been proposed to prevent cross-contamination of patients through the use of a stethoscope.

However, past attempts to incorporate antimicrobial activity have not retained desired levels of acoustic sensitivity or antimicrobial action. Thus, there may be a need for a stethoscope diaphragm that exhibits antimicrobial activity while retaining its acoustic sensitivity.

### SUMMARY

The present embodiments relate to a medical device useful for enhancing microbial protection while retaining desired acoustic sensitivity.

In some embodiments, a medical device element is described, the device element comprising an acoustically transmissive element having a sound transmission with less than 5 decibels of transmission loss, said acoustically transmissive element having a first transmissive element side and a second transmissive element side. Some embodiments include an antimicrobial element, the antimicrobial element disposed on or within the first side of the acoustically transmissive element such that the acoustic transmission of the medical device is within 0.5 decibels of the acoustic transmission loss of the acoustically transmissive element when substantially free of the antimicrobial element, the medical device element having a device first side and a device second side, wherein the device first side contacts a patient body surface.

In some embodiments, the acoustically transmissive element comprises a substrate having a first acoustically transmissive element side and second acoustically transmissive element side, and wherein the antimicrobial element comprises a layer disposed upon the first acoustically transmissive element side.

In some embodiments, the acoustically transmissive element comprises an acoustically transmissive element matrix material, wherein the antimicrobial element is disposed within said acoustically transmissive matrix material. In some embodiments, the matrix material comprises a portion adjacent to the patient interface surface, wherein the antimicrobial element is disposed within the portion adjacent to the patient interface surface.

These and other embodiments are described in greater detail below.

### BRIEF DESCRIPTION OF THE DRAWINGS

FIGS. 1A-F are a schematic of a stethoscope diaphragm described herein.

FIGS. 1B-F are schematics of an embodiment of a stethoscope diaphragm described herein.

FIG. 2 is a schematic of a stethoscope.

FIG. 3 is a transmitted amplitude-time waveform of a normal human heartbeat through a commercially available stethoscope.

FIG. 4 is a transmitted amplitude-time waveform of a normal human heartbeat through an embodiment of a stethoscope diaphragm described herein.

FIG. 5 is a transmitted amplitude-time waveform of a normal human heartbeat through another embodiment of a stethoscope diaphragm described herein.

FIG. 6 is a transmitted amplitude-time waveform of a normal human heartbeat through a comparative stethoscope diaphragm.

FIG. 7 is a graph of antimicrobial activity of an embodiment of a stethoscope diaphragm described herein.

### DETAILED DESCRIPTION

Some embodiments disclosed herein can reduce the transfer of microbial pathogens between/from patients and/or healthcare workers.

The term acoustically transmissive material includes its common meaning in the field and includes a material that can transmit sound from one surface of the material to different surface of the material. In some embodiments, the material can transmit sound without significant transmission loss, such as about 20 decibels or less, about 15 decibels or less, about 12 decibels or less, about 10 decibels or less, about 5 decibels or less, about 1 decibel or less, or about 0.5 decibels or less, of transmission loss. In some embodiments, the material can transmit sound without significant transmission loss, for example, a decrease in the maximum amplitude transmitted relative an uncovered or uncoated transmissive element. In some embodiments, the material can transmit sound without significant transmission loss, for example, a decrease in the average amplitude transmitted relative to an uncovered or uncoated transmissive element. In some embodiments, the decrease in amplitude can be compared using relative analogous peaks. For example, if the uncovered or uncoated material displays seven peaks and the covered or coated material also displays seven peaks in an amplitude-time waveform, then the respective seventh peak of each amplitude-time waveform can be compared.

In some embodiments, a medical device element is comprised of an acoustically transmissive element having a sound transmission with less than 5 decibels of transmission loss. In some embodiments, the acoustically transmissive element has a first transmissive element side, a second transmissive element side, and an antimicrobial element. In some embodiments, the antimicrobial element may be disposed on or within the first side of the acoustically transmissive element, such that the acoustic transmission of the medical device is within 0.5 decibels of the acoustic transmission loss of the acoustically transmissive element when substantially free of the antimicrobial element, the medical device element having a device first side and a device second side, wherein the device first side contacts a patient body surface.

In some embodiments, the acoustically transmissive element may comprise a substrate having a first acoustically transmissive element side and second acoustically transmissive element side, wherein the antimicrobial element may comprise a layer disposed upon the first acoustically transmissive element side.

In some embodiments, the acoustically transmissive element may comprise a matrix material, and wherein the antimicrobial element may be disposed within said matrix material.

In some embodiments, the matrix material may comprise a portion adjacent to a patient interface surface, wherein the antimicrobial element may be disposed within the portion adjacent to the patient interface surface.

In some embodiments, the medical device can be an externally contacting device, wherein the device contacts an exterior surface of the mammalian or human body. Some embodiments of an exterior contacting device, include, but are not limited to, stethoscope diaphragms, echocardiogram devices, acoustic probes based on the Doppler effect, ultrasound wands, transducers and probes, etc.

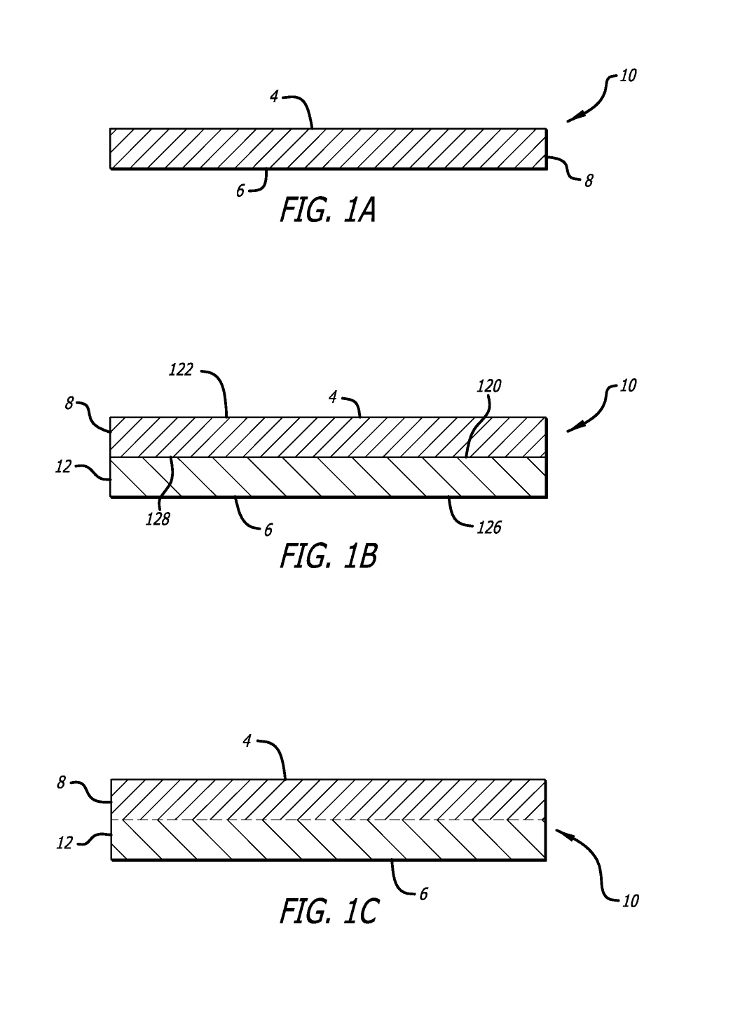

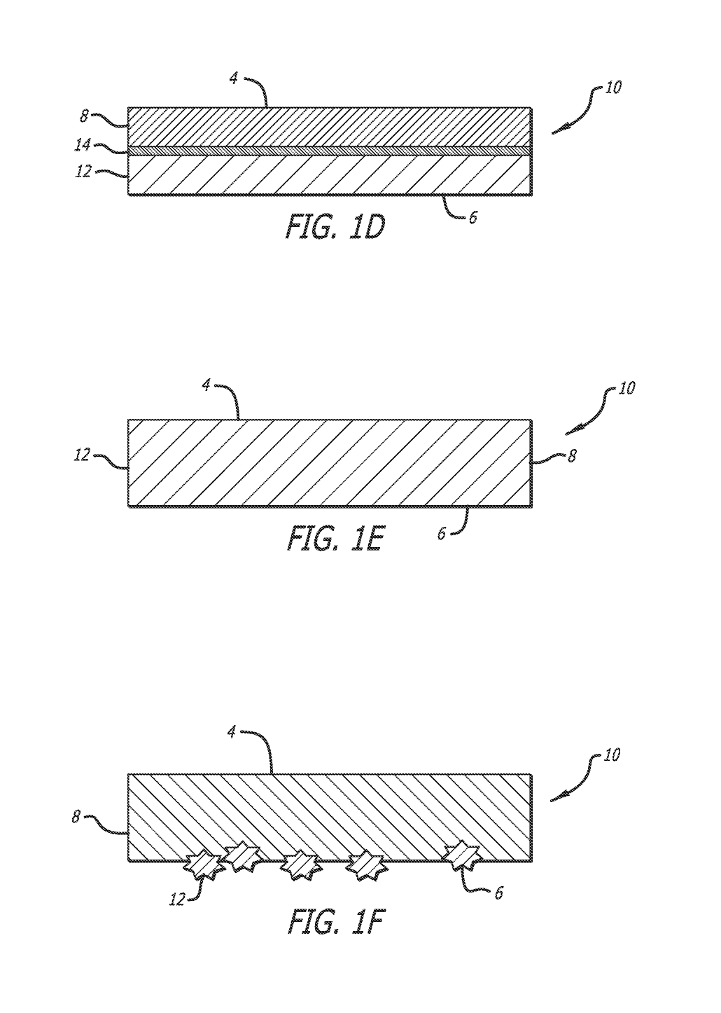

Referring now to FIG. 1A, a medical device element 10 is shown having an acoustically transmissive element 8 having a first transmissive element side or contact surface 6 and a second transmissive element side or surface 4. In some embodiments, the acoustically transmissive element comprises a substrate. In some embodiments, the acoustically transmissive element comprises a matrix. In some embodiments, the contact surface can contact an interior body surface. In some embodiments, the contact surface can contact an external body surface or part.

Referring now to FIGS. 1B-1E, a medical element 10 comprising a medical element substrate 8 is shown in combination with an antimicrobial element 12, wherein the medical element has a first or patient contact surface 6 and a second surface 4. In FIG. 1B, a medical element 10 is shown with a separate acoustically transmissive element or substrate 8, with a first substrate surface 120 and a second substrate surface 122, and an antimicrobial element 12, with a first antimicrobial element surface 126 and a second antimicrobial element surface 128. Substrate 8 and antimicrobial element 12 are conjoined to form antimicrobial medical element 10. In some embodiments, the substrate has a first transmissive element or substrate side and a second transmissive element or substrate side. In some embodiments, the antimicrobial element comprises a layer disposed upon the first acoustically transmissive element side. In this embodiment, a discernible boundary between the two elements is perceived. In this embodiment, the contact surface 6 coincides with the first antimicrobial element surface 126.

In FIG. 1C, medical element substrate 8 and antimicrobial element 12 are conjoined to form antimicrobial element 10. In this embodiment, an indiscernible boundary, as indicated by the dotted lines, may exist between the two elements. In some embodiments, the medical element 8 substantially comprises the matrix substrate material, and the antimicrobial element 12 substantially comprises the antimicrobial substrate material. In some embodiments, there exists a gradient or material transition between portions consisting essentially of, or containing at least 50% matrix material, to portions consisting essentially of, or containing at least 50% of the antimicrobial element material. In some embodiments, a gradient of material transitions from portions comprising more of the medical device matrix material to portions comprising more of the antimicrobial element material. In some embodiments, the device matrix material comprises a portion adjacent to and/or forming a portion of the patient interface surface. In some embodiments, the antimicrobial element material is disposed within the portion adjacent to and/or a portion of the patient interface surface. In some embodiments, the antimicrobial material is disposed within the matrix material. In some embodiments, the antimicrobial material has been diffused into the medical element matrix material.

In FIG. 1D, medical element substrate 8 and antimicrobial element 12 are joined by adhesive layer 14. In some embodiments, the adhesive layer can be located substantially entirely over the interface between medical element 8 and antimicrobial element 12. In some embodiments, the adhesive layer can be over a portion of the surface interface between medical element 8 and the antimicrobial element 12. In some embodiments, the portion of the surface interface without the adhesive layer maintains acoustical communication with antimicrobial element 12. In some embodiments, the antimicrobial element portion without the adhesive layer is in physical contact with the medical element.

In FIG. 1E, medical element substrate 8 and antimicrobial element 12 are combined to form antimicrobial element 10.

In FIG. 1E, antimicrobial element 10 can comprise a composite element, wherein the material of an medical device substrate 8 and antimicrobial element 12 are blended, dispersed, or alloyed to form antimicrobial element 10. In FIG. 1F, antimicrobial element 10 comprises a composite element, wherein antimicrobial elements 12 are separate plural islands of material disposed in the surface of medical element 8.

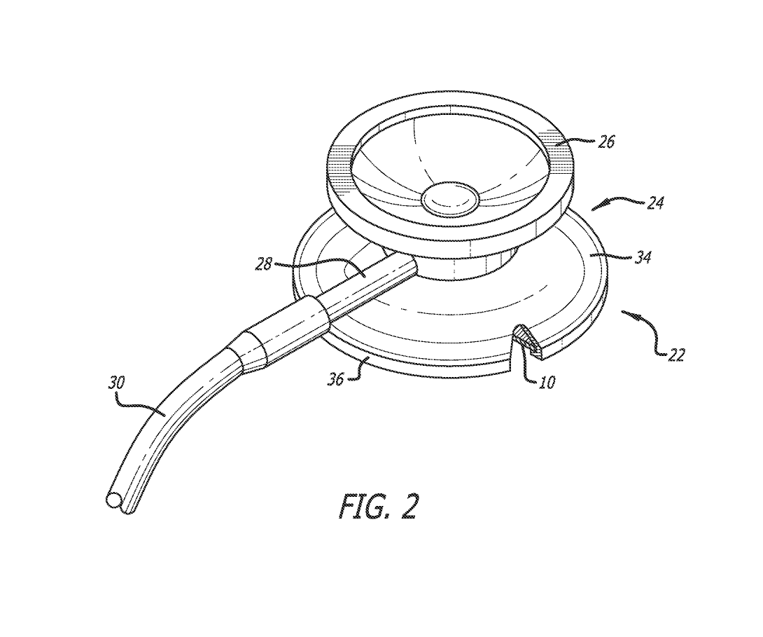

Referring now to FIG. 2, medical device element 10 is shown in relation to a stethoscope head 22. In one embodiment, the medical device element can be a modified stethoscope diaphragm, which can be used with any stethoscope provided it is structurally figured to closely fit the diameter of the diaphragm.

Medical element 8 in the form of a stethoscope can comprise a head 22. Head 22 can comprise a diaphragm portion 24, a bell portion 26, and a tubular outlet member 28 containing an air column. A flexible hose 30 connects the outlet 28 to a hinge that in turn is attached to the binaural earpiece members (not shown). Diaphragm portion 24 is formed of a flat cup 34 on which is mounted a semi-rigid medical element 10, which can include a diaphragm portion or substrate, held in place by a circular ring 36.

In some embodiments, the diaphragm can comprise a matrix or substrate of an acoustically transmissive material. In some embodiments, an acoustically transmissive material transmits at least about 70%, at least about 80%, at least about 90%, or at least about 95% of the acoustic wave energy from one surface to the another surface. In some embodiments, an acoustically transmissive material loses less than about 1 dB, less than about 2 dB, less than about 3 dB, less than about 4 dB, less than about 7 dB, or less than about 10 dB of sound from one side to the other side. In some embodiments, the matrix can comprise epoxy and fiberglass. A suitable matrix material can be the material commercially available as a Littman Cardiology III diaphragm (3M, Minneapolis, Minn., USA).

In some embodiments, a photocatalytic element can be substantially acoustically transparent. In some embodiments, the acoustic transmission of the diaphragm further comprising a photocatalytic element is within at least about 0.25 decibels, about 0.5 decibels, about 0.75 decibels, about 1.0 decibels, about 1.5 decibels, about 2.0 decibels, about 2.5 decibels, about 3.0 decibels, about 3.5 decibels, about 4.0 decibels, about 4.5 decibels, and/or about 5.0 decibels of the acoustic transmission loss by the diaphragm when substantially free of the antimicrobial element. In some embodiments, the acoustic transmission of the photocatalytic material is within at least about 0.25 decibels, about 0.5 decibels, about 0.75 decibels, about 1.0 decibels, about 1.5 decibels, about 2.0 decibels, about 2.5 decibels, about 3.0 decibels, about 3.5 decibels, about 4.0 decibels, about 4.5 decibels, and/or about 5.0 decibels of the acoustic transmission loss by the diaphragm when substantially free of photocatalytic material.

In some embodiments, the antimicrobial element comprises an inorganic material. In some embodiments, the inorganic material can be, but is not limited to silver, silver compounds (e.g., silver dihydrogen citrate (SDC)), copper, copper alloys, copper compounds, triclosan, halogen releasing compounds, and selenium containing compounds. In some embodiments, the inorganic material is substantially free of arsenic, silver, tin, heavy metals, polychlorinated phenols, and/or any combinations thereof.

In some embodiments, the antimicrobial element is photocatalytic. In some embodiments, the antimicrobial element is photocatalytic. In some embodiments, the photocatalytic element can effect at least a 0.5 log reduction in an exposed bacterial population, at least a 0.75 log reduction in the exposed bacterial population, at least a 1.0 log reduction in the exposed bacterial population, 1.5 log reduction in the exposed bacterial population, 2.0 log reduction in the exposed bacterial population, 2.5 log reduction in the exposed bacterial population, and/or at least a 3.0 log reduction in the exposed bacterial population. In some embodiments, any or all of the aforedescribed log reductions can be effected in about 3 minutes, in about 5 minutes, in about 10 minutes, in about 15 minutes, in about 20 minutes, in about 30 minutes, in about 45 minutes, in about 1 hour after initial exposure of the bacteria/bacterial population to the antimicrobial element. In the above discussion, “log reduction” refers to commonly accepted terminology in microbiology for describing the reduction by orders of magnitude of viable microbial population. For instance a 1.0 log reduction corresponds to a 90% reduction and a 2 log reduction corresponds to a 99% reduction. In some embodiments, the bacterial population can comprise E. coli.

In some embodiments, the E. coli strain can be ATCC 8739. In some embodiments, the bacterial population can comprise S. aureus. In some embodiments, the S. aureus strain can be ATCC 6538. In some embodiments, the microbial population can comprise Staphylococcus aureus, Escherichia coli., Enterobacter aerogenes, Pseudomonas aeruginosa, Feline calicivirus (analog for Norovirus), Acinetobacter baumanii, Moraxella osloensis, Bacillus subtilis, Bacillus sphaericus, Aspergillus brasiliensis, Rhodotorula mucilaginosa, Methicillin-resistant Staphylococcus aureus (MRSA), Enterococcus faecalis (vancomycin-susceptible), Vancomycin-resistant Enterococcus faecium (VRE), Heterogeneously vancomycin-intermediate Staphylococcus aureus (hVISA), Vancomycin-intermediate Staphylococcus aureus (VISA), Pseudomonas aeruginosa with AmpC overexpression and OprD deletion, Extended-spectrum beta-lactamase (ESBL)-producing Escherichia coli (CTX-M-1 and CTX-M-9), ESBL-producing Klebsiella pneumoniae (CTX-M-1 and SHV-like), AmpC-producing Enterobacter cloacae, KPC-2-producing Klebsiella pneumoniae from Brooklyn, N.Y., KPC-3-producing Klebsiella pneumoniae from Houston, Tex. and NDM-1-producing Klebsiella pneumonia.

In some embodiments, the antimicrobial element comprises at least one inorganic oxide. Suitable photocatalysts include, but are not limited to, loaded and/or unloaded, doped and/or undoped forms of titanium dioxide (TiO₂), tungsten oxide (WO₃), strontium titanate (SrTiO₃), SnTi(C,N,O)₂, cerium dioxide (CeO₂), tin oxide (SnO₂), copper(I) oxide (Cu₂O), copper(II) oxide (CuO) and/or combinations thereof. In some embodiments, the photocatalytic material is a plural phase composite of photocatalytic materials. In some embodiments, the photocatalytic material can be anatase, rutile, wurtzite, spinel, perovskite, pyrocholore, garnet, zircon, and/or tialite phase material or mixtures thereof. Each of these options is given its ordinary meaning as understood by one of ordinary skill in the art. Comparison of an x-ray diffraction pattern of a given standard and the produced sample is one of a number of methods that may be used to determine whether the sample comprises a particular phase. Exemplary standards include XRD spectra provided by the National Institute of Standards and Technology (NIST) (Gaitherburg, Md., USA) and/or the International Centre for Diffraction Data (ICDD, formerly the Joint Committee on Powder Diffraction Standards [JCPDS]) (Newtown Square, Pa., USA).

In some embodiments, the plural phase photocatalytic materials comprise anatase phase and rutile phase compounds. In some embodiments, the plural phase photocatalytic materials include titanium oxides. In some embodiments, the anatase phase is about 2.5% to about 97.5%, about 5% to about 95%, and/or about 10% to about 90%, or any percentage bounded by or between any of these percentages; and the rutile phase is about 97.5% to about 2.5%, about 95% to about 5%, and/or about 10% to about 90%, or any percentage bounded by or between any of these percentages. A non-limiting example of a suitable material includes a TiO₂ mixture sold under the brand name P25 (about 83% Anataste TiO₂+about 17% Rutile TiO₂) sold by Evonik (Parissipany, N.J., USA)).

In some embodiments, suitable photocatalysts include composite multivalence metal loaded oxides. In some embodiments, multivalence metal loaded oxides comprise a p-type compound/composition/element in electrochemical communication with an n-type compound/composition/element. Some useful embodiments related to multivalence metal loaded oxides and information related to materials that may be useful in p-type and n-type compositions are described in U.S. patent application Ser. No. 13/840,859, filed Mar. 15, 2013 (United States Patent Application Publication 2014/0271916, published on Sep. 18, 2014), and U.S. Provisional Application No. 61/835,399, filed Jun. 14, 2013, which are each hereby incorporated by reference in their entireties. Some useful embodiments related to materials that may be useful in photocatalytic compositions may be described in U.S. Provisional Application No. 61/585,732; U.S. patent application Ser. No. 13/741,191, filed Jan. 14, 2013 (United States Patent Application Publication 2013/0192976, published Aug. 1, 2013), U.S. patent application Ser. No. 13/738,243, filed Jan. 10, 2013 (United States Patent Application Publication 2013/0180932, published Jul. 18, 2013); U.S. patent application Ser. No. 13/840,859, filed Mar. 15, 2013; U.S. Provisional Application No. 61/835,399, filed Jun. 14, 2013, and U.S. Provisional Application No. 61/843,266, filed Jul. 5, 2013, which are each hereby incorporated by reference in their entireties.

Referring again to FIG. 1F, in some embodiments, the photocatalysts need not be a complete film or layer over the matrix or substrate. For example, the photocatalysts could include a plurality of nanoparticles, microparticles, nanostructures, or microstructures that may be dispersed on, but do not necessarily entirely cover, the surface upon which the photocatalysts could be deposited. In some embodiments, an external contacting surface may have a surface comprising a plurality of irregularly arranged protrusions, particles, or aggregates thereof. In some embodiments, an external contacting surface may have a surface comprising a plurality of regularly arranged protrusions, particles, or aggregates thereof.

The protrusions or particles may be nanoprotrusions, including nanoprotrusions having one or more dimensions in the nanometer to micron range. For example, nanoprotrusions or nanoparticles may have: an average×dimension of about 400 nm, about 500 nm, about 1000 nm, about 1500 nm, about 2000 nm, about 2500 nm, about 3000 nm, or any value in a range bounded by, or between, any of these lengths; an average y dimension of about 50 nm, about 100 nm, about 300 nm, about 500 nm, about 700 nm, about 1000 nm, about 1200 nm, about 1500 nm, about 1800 nm, about 2000 nm, or any value in a range bounded by, or between, any of these lengths; and/or an average z dimension of about 10 nm, about 30 nm, about 50 nm, about 70 nm, about 90 nm, about 100 nm, or any value in a range bounded by, or between, any of these lengths. In some embodiments, at least one particle in the film, or average of the particles in the film, may have an x dimension, a y dimension, or a z dimension of: about 5 nm, about 0.01 μm, about 0.02 μm, about 0.05 μm, about 0.1 μm, about 0.5 μm, about 1 μm, about 2 μm, about 5 μm, about 10 μm, about 20 μm, about 50 μm, about 100 μm, about 150 μm, about 200 μm, about 500 μm, about 1000 μm, about 5 nm to 200 about 1000 μm or about 1000 μm, or any length in a range bounded by, or between, any of these values.

In some embodiments, the antimicrobial element does not include a binder and is inputted directly into or is integral with the substrate and/or matrix material.

In some embodiments, the antimicrobial element comprises an antimicrobial element matrix material and an antimicrobial compound. In some embodiments, the matrix material can be a binder material. In some embodiments, a weight ratio of binder to photocatalytic material is about 0.5 to 2.0 parts binder (weight %) to about 1 part (weight %) photocatalytic material. In some embodiments, the binder material is a polymeric compound. In some embodiments, the binder material is a silicone resin. In some embodiments, the silicone resin is a silicone alkyd resin, a silicone epoxy resin, a silicone acrylic resin, or a silicone polyester resin. In some embodiments, the silicone resin is a silicone polyester resin. In some embodiments, the suitable silicone polyester resin is a commercially available product, e.g., KR5230 and/or KR5235 (Shin-Etsu Chemical Co., Ltd, Tokyo, Japan).

The antimicrobial element can comprise materials disclosed in U.S. Provisional Application 61/899,423, filed Nov. 4, 2013, which is incorporated by reference in its entirety.

In some embodiments, the substrate can form a patient interface surface. A patient interface surface refers to a surface of a device that may be in direct or indirect contact with the skin of a human during ordinary operation of that device. In some embodiments, the acoustically transmissive substrate and/or matrix material is acoustically transmissive.

In some embodiments, the acoustically transmissive element is a diaphragm. In some embodiments, the diaphragm comprises an acoustically transparent matrix/substrate and an antimicrobial element layer contacting the substrate. In some embodiments, the antimicrobial element is formed as a layer on the surface of the substrate. In some embodiments, the antimicrobial element comprises an acoustically transparent matrix and an inorganic photocatalytic material.

In some embodiments, the acoustically transparent matrix comprises a material that is acoustically integral with the substrate. In some embodiments, the acoustically transparent matrix comprises a material that is not acoustically integral with the substrate. In some embodiments, the material includes a diaphragm comprising a composite material, wherein the composite material comprises an inorganic photocatalytic material and an acoustically transparent substrate material. In some embodiments, the material includes a stethoscope diaphragm comprising an acoustically transparent substrate and an antimicrobial layer that is bound to the diaphragm surface. In some embodiments, the above-described bound layers are polymerically bound, e.g., by using a polymer adhesive; using a polymer and monomer mixture, then curing the same; etc. In some embodiments, the bound layers are bound by thermally heating the materials to fluidize them and subsequently cooling and binding them together. In some embodiments, the adhesion of the layers to each other can be evaluated by known adhesion test methods, for example, following the procedures described in ASTM-D3359. In some embodiments, the percentage of the antimicrobial layer removed is less than about 35%, less than about 20%, less than about 15%, less than about 10%, less than about 5% using the procedures described in ASTM-D3359. In some embodiments, the layers are characterized by an adhesion test value of at least 0 B, at least 1 B, at least 2 B, at least 3 B, at least 4 B, at least 5 B, and/or at least 6 B using the procedures described in ASTM-D3359. In some embodiments, the adhesion of the photocatalytic coating is evaluated by known hardness tests. In some embodiments, the layers are characterized by a hardness test of at least 2 H, of at least 3 H, of at least 4 H. Hardness of the photocatalytic element/coating can be evaluated by known hardness determination methods, for example, the procedures described in ASTM-3363.

In some embodiments, the antimicrobial element is formed as a layer by vapor deposition such as chemical vapor deposition (CVD) or physical vapor deposition (PVD); laminating, pressing, rolling, soaking, melting, gluing, sol-gel deposition, spin coating; dip coating; bar coating; slot coating; brush coating; sputtering; thermal spraying including flame spray, plasma spray (DC or RF); high velocity oxy-fuel spray (HVOF) or atomic layer deposition (ALD); cold spraying or aerosol deposition.

In some embodiments, the photocatalytic composition is partially embedded into the surface of the substrate by direct loading. Some substrates are formed by creating a mixture of a polymer and a solvent. Then the photocatalytic composition is directly loaded into the mixture, creating a slurry. The resulting slurry is formed into the substrate so that the photocatalytic composition is integral with the substrate. In some embodiments, the photocatalytic composition is an integral part of the surface of the resulting substrate. In some embodiments, the photocatalyst composition substantially covers the substrate. In some embodiment, the photocatalyst composition contacts or covers at least about 75%, at least about 85%, at least about 95%, or about 100% of the substrate surface.

In some embodiments, the photocatalytic composition is partially embedded into the surface of the substrate by particle transfer. A suitable method for achieving this can be as described in U.S. Provisional Application 61/898,980, filed Nov. 1, 2013, and JP2014-113003, filed May 30, 2014, which are incorporated by reference in their entirety. In some embodiments, the photocatalytic composition is partially embedded in the surface of the substrate by chemical etching. A suitable method for achieving this can be as described in U.S. Provisional Application 61/946,611, filed Feb. 28, 2014, U.S. Provisional Application 61/931,387, filed Jan. 24, 2014, U.S. Provisional Application 62/007,489, filed Oct. 30, 2013, and JP2014-113003, filed May 30, 2014, which are incorporated by reference in their entirety.

In some embodiments, the photocatalytic element comprises a photocatalytic material and a polymer film. In some embodiments, the polymer film can be a PET film with an adhesive backing so that the substrate may be affixed to medical equipment.

In some embodiments, the medical element further comprises a visual indicator to illustrate the remaining efficacy of the antimicrobial photocatalytic composition, so a user will know when to replace the device for example a color change or fading of text or symbols. In some embodiments, the photocatalytic element comprises the visual indicator.

### EXAMPLES

The benefits of the medical device elements described herein are further shown by the following examples, which are intended to be illustrative of the embodiments of the disclosure, but are not intended to limit the scope or underlying principles in any way.

### Example 1A

### Manufacturing the Photocatalytic Element (Ex-1)

### Example (1AA)

### Copper Sputtered on Diaphragm

A copper metal target was provided as copper source material and placed on the target platform (cathode side) within the vacuum chamber of a Cressington 108 auto sputtering apparatus. A 5 cm circle of a stethoscope diaphragm (Littman Cardiovascular III diaphragm, 3M, Minneapolis, Minn., USA) was placed on the anode side. The sputtering apparatus was provided with the following parameters: driving current about 0.1 mA and a pressure of 1 Torr. Argon gas was introduced into the vacuum chamber for deposition of about 20 sec. The substrate temperature was at room temperature.

### Example (1AB)

### Photocatalytic (CuxO/Plural Phase TiO₂) Coating on Diaphragm)

A commercially available Littman stethoscope diaphragm (Littman Cardiology III, 3M, Minneapolis, Minn., USA) was used as a substrate for photocatalytic coating. The diaphragm was cleaned with a sequence of soap and water, acetone, and methanol, and then dried.

A binder solution containing 10 wt % silicone modified polyester resin was made by mixing a modified silicon polyether resin (sold under the brand designation, “KR-5230”, by ShinEtsu Silicones, JAPAN) with PGMEA (Propylene Glycol Monomethyl Ether Acetate, reagent>99.5%, Sigma-Aldrich). The mixing was conducted with a planetary centrifugal mixer (THINKY AR-310) at about 2000 rpm for 2 min for mixing and then at about 2200 rpm for about 1 min for defoaming.

To make a coating suspension, one part of photocatalytic powder was mixed with 10 parts of the aforementioned binder solution. The photocatalytic powder comprises copper loaded plural phase titanium oxide that increases the light absorption in visible light range. The nominal copper content in the photocatalytic material was 1 wt %. 1 gm of photocatalytic powder was dispersed in 10 gm of binder solution (10% binder in 90% solvent) by keeping the glass vial containing the mixture in a sonication bath for about one hour. The obtained suspension was passed through an in-line filter with stainless steel screen having openings of 30 micrometers.

The coating of the substrate (Ex-2) was preformed on the prepared stethoscope diaphragm substrate by spin coating with a spin coater (SCS 6800 series, Specialty Coating System) at about 1200 rpm for about 20 sec.

The diaphragm substrate with photocatalytic coating was dried at ambient atmosphere at 110° C. for about 1 hour.

### Example (1AC)

### Photocatalytic (CuₓO/Plural Phase TiO₂) Coating on PET then Attached to Diaphragm

An additional example (Ex-3) was prepared in a manner similar to Ex-2, except that the photocatalytic coating was formed on a prepared PET substrate instead of directly on a stethoscope diaphragm by tape casting with use of a doctor blade and a tape caster (AFA-II, MTI Corporation). The gap of the doctor blade was kept in the range of 3 mil to 20 mils (one mil equals to 1/1000 inch or 25.4 micrometers). The PET substrate with photocatalytic coating was dried at ambient atmosphere at 110° C. for about 1 hour. The coated PET sheet was cut to the same diameter as the stethoscope diaphragm. Nitto brand double-sided adhesive tape (AS-1902P12 (Nitto Denko, Osaka, JP) was applied to the surface of the PET sample opposite the coating and the PET coated sheet was attached to the stethoscope diaphragm

### Example 2

### Adhesion/Hardness Testing

Adhesion of Photocatalytic coating was evaluated by following the procedures described in ASTM-D3359.

Coating hardness was evaluated using the procedures described in ASTM-3363.

TABLE 1

| | | Photocatalytic/ | | | |
|---|---|---|---|---|---|
| | Binder | binder | Photocatalytic | Binder | |
| | solution | solution | loading | loading | |
| Sample | (wt %) | (w/w) | (vol %) | (vol %) | Adhesion |
| Exam- | 20 | 1/50 | 2.6 | 97.4 | 4B-5B |
| ple A | | | | | |

Removed percentage: 0 B>65%; 1 B: 35-65%; 2 B: 15-35%; 3 B: 5-15%; 4 B<5%; 5 B:0%

### Example 3

### Acoustic Testing

Digital audio recordings of normal human heart beat sounds were played from an Apple iPhone 5 and reproduced by a Stethoscope Sounder (A.C.T.N.T. Healthcare Services) connected to the analog audio-out jack of the iPhone. The normal heart beat sound file was downloaded from www.thinklabsmedical.com/sound-library.html. The diaphragm of a commercial stethoscope (Littmann Cardiology III, 3M, USA) was placed on the Stethoscope Sounder with a 300 g lab weight on top of the stethoscope bell to simulate hand pressure used during regular auscultation. One of the ear pieces of the stethoscope was fitted with an electret microphone (RadioShack 33-3013) for capturing the transmitted sound. The output of the electret microphone was captured via a Windows 7 PC running Adobe Audition CS6 software. The other stethoscope earpiece wave was stuffed with polyurethane foam to dampen external noise while letting the sound waves from the diaphragm escape without creating back pressure. Diaphragms described in Example 1, etc. above, with and without modifications, were tested in this manner and waveforms were analyzed using Adobe Audition CS6 and in Matlab. The transmission through the diaphragm of a pink noise file generated using Adobe Audition CS 6 was used for detailed studies.

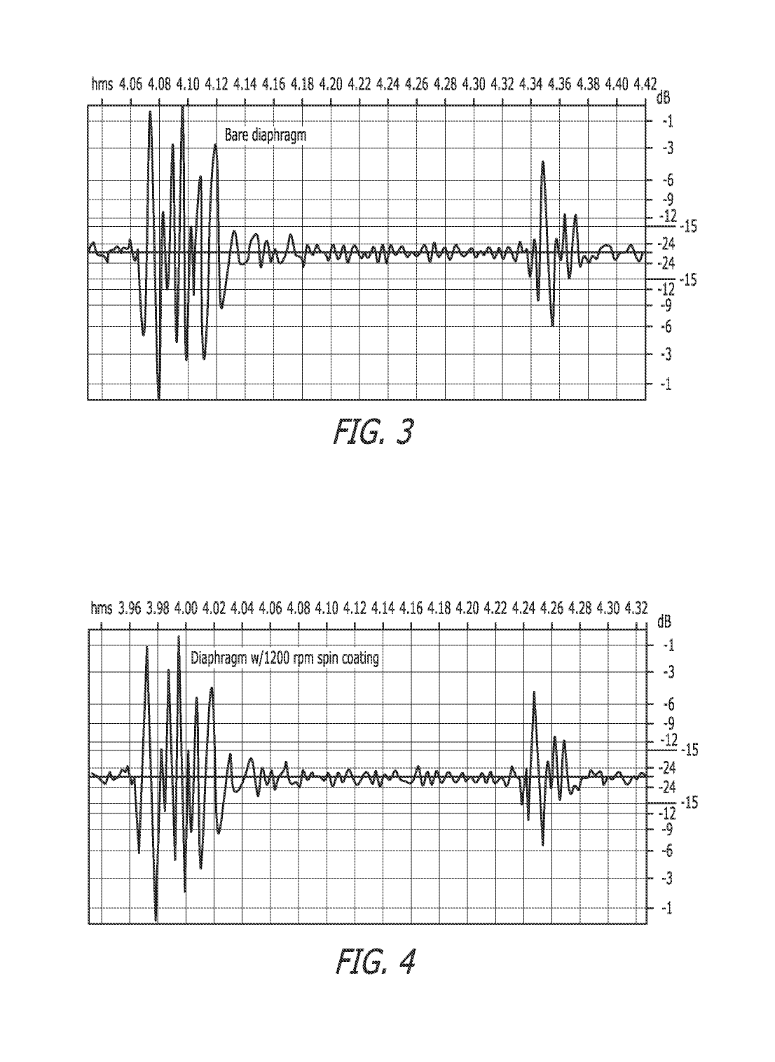

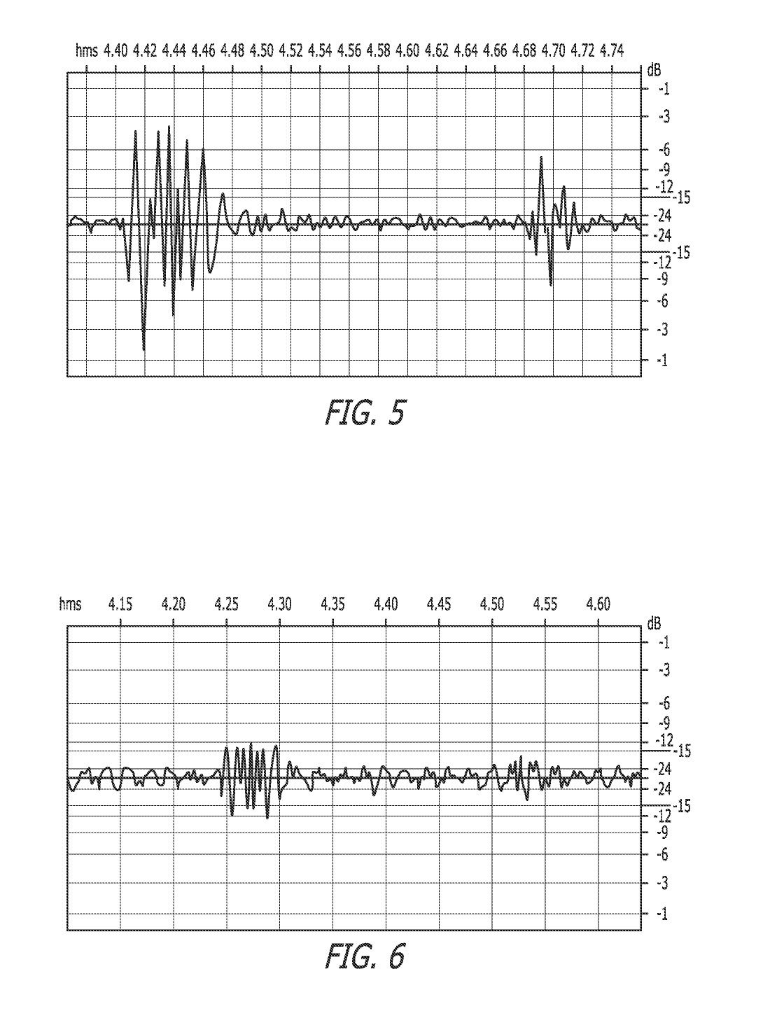

The results are shown in FIGS. 3-6. FIG. 3 shows the sound wave depicted by an uncoated/bare diaphragm. FIG. 4 shows the sound wave depicted by the coated diaphragm (Ex-1) made as described in Example 1AA (sputtering) above. FIG. 5 shows the sound wave depicted by the coated PET/diaphragm (Ex-3) made as described in Example 1AC (tape cast CuₓO)/P25 diaphragm) above. FIG. 6 shows the sound wave depicted by a comparative silver infused hard plastic diaphragm cover (Stethocap, weekly use diaphragm cover, model number 2PTFTA, Algonquin, Ill., USA). FIG. 6 shows a different (lesser) acoustical response than that of the modified diaphragm, made in accordance to Example 1AA (sputtering) (FIG. 4) and/or Example 1AC (tape case coating) (FIG. 5), which show substantially the same acoustical response as the untreated/uncoated diaphragm (FIG. 3).

Antibacterial performance was evaluated by following the procedures.

The respective diaphragms were placed in a glass dish with a water soaked filter paper for maintaining moisture, and glass spacers were inserted between the substrate and the filter paper to separate them.

E. coli (ATCC 8739) was streaked onto a 10 cm diameter petri dish containing about 20 ml of LB (lysogeny broth/luria broth) agar, and incubated at about 37° C. overnight. For each experiment, a single colony was picked to inoculate about 3 mL nutrient broth, and the inoculated culture was incubated at about 37° C. for about 16 hours to create an overnight culture (˜109 cells/mL). A fresh log-phase culture of the overnight culture was obtained by diluting the overnight culture ×100, inoculating another 5 cm petri dish with LB agar and incubating at about at 37° C. for about 2.5 hr. The fresh culture was diluted 50× with 0.85% saline, which gave a cell suspension of about 2×10⁶ cells/mL. 50 μL of the cell suspension was pipetted onto each deposited glass substrate. A sterilized (in 70% and then 100% EtOH) plastic film (20 mm×40 mm) was placed over the suspension to spread evenly under the film. The specimen was kept in the dark (CuxO₂-Dark) or then irradiated under blue LED light (455 nm, 10 mW/cm²) (CuO₂-light). At a chosen time point, e.g., 30 min/60 min increments, the specimen was placed in 10 mL of 0.85% saline and vortexed to wash off the bacteria. The wash off suspension was retained, then serially diluted using 0.85% saline, and then plated on LB agar and incubated at about 37° C. overnight to determine the number of viable cells in terms of CFU/Specimen.

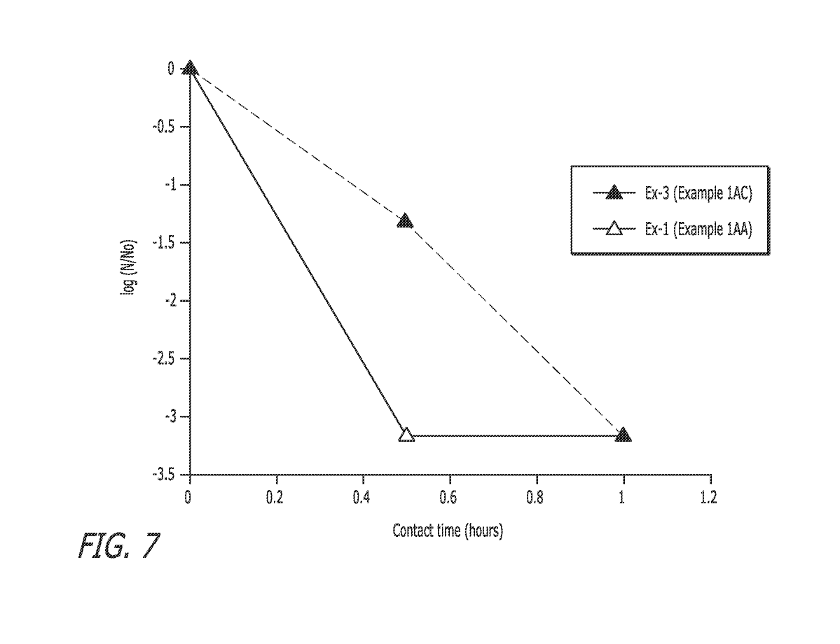

FIG. 7 shows the antibacterial (E. Coli) performance of photocatalytic coated PET attached to a diaphragm made as described in Example 1AC (Ex-3) and a diaphragm made as described in Example 1 (sputtering). FIG. 7 shows the diaphragm made as described in Example 1AA (Ex-1) provided a log 3 decrease in the bacterial population after 30 minutes exposure to the antibacterial element. FIG. 7 also shows the diaphragm made as described in Example 1AC (Ex-3) (photocatalyst coated PET substrate) provided a log 1.3 decrease in the bacterial population after 30 minutes exposure to the respective antibacterial element and a log 3 decrease in the bacterial population after 60 minutes of exposure to the antibacterial element.

### Example 4

### Photocatalyst (CuₓO/TiO₂) Coating on PET Substrate

A commercially available PET (polyethylene terephthalate) film (Eplastics Inc. San Diego, Calif. USA) with a thickness of about 120 micrometers (microns) was used as a substrate for a photocatalytic coating. The substrate was cut into paper size. The cut PET substrate was cleaned with acetone and then dried.

A binder solution containing 10 wt % uv curable hard coat was made by mixing about 1 g uv-curable acrylate binder (sold under the brand designation, Unidic17806, by DIC corporation, JAPAN), about 24 mg of a photoinitiator (sold under the brand designation Irgacure 907) and about 10 g Cyclopentanone (reagent>99.5%, Sigma-Aldrich). The mixing was conducted with planetary centrifugal mixer (THINKY AR-310) at about 2000 rpm for 2 min for mixing and then at about 2200 rpm for about 1 min for defoaming.

To make a coating suspension, one part of CuxO/P25 photocatalytic powder (about 0.2 g) by weight was mixed with 5 part, by weight, of binder solution (10 wt % urethane acrylate dissolved in cyclopentanone). The photocatalytic powder was made according to that described in U.S. patent application Ser. No. 13/840,859, filed Mar. 15, 2013; and U.S. Provisional Application 61/835,399, filed Jun. 14, 2013; and U.S. patent application Ser. No. 13/741,191, filed Jan. 14, 2013 (United States Publication No. 2013/0192976, published Aug. 1, 2013). The photocatalytic-cat powder comprises copper oxide loaded titanium oxide doped with carbon, nitrogen and tin to increase the light absorption in visible light range. The nominal copper content in P-cat was 1 wt %. 0.2 gm of photocatalytic powder was dispersed in the binder solution (about 1 gm, 10% solution) by keeping the glass vial containing the mixture in a sonication bath for about half hour followed by probe sonication for about 20-30 mins. The obtained suspension was passed through a filter with opening of 5 micrometers.

Prior to coating, the cleaned PET substrate was subject to corona discharge treatment to increase the hydrophilicity of the substrate surface for good wettability of the coating suspension. A corona treatment apparatus (TEWC-4AX, KASUGA DENKI Inc. JAPAN) was used at discharge power of 100 W and scan speed of 0.5 m/sec for two scans.

The coating of the substrate (Test 1-8) was performed on the prepared PET substrate by tape casting with the use of a doctor blade and a tape caster (AFA-II, MTI Corporation) by the method described in U.S. Pat. No. 8,283,843, filed Jan. 28, 2011, issued Oct. 9, 2012. The gap of the doctor blade was kept in the range of 3 mils to 20 mils (one mil equals 1/1000 inch or 25.4 micrometers). The PET substrate with a photocatalytic coating was dried at ambient atmosphere and then preheated at 90 to100° C. for about 2 min, then uv cured under Dymax UV conveyor system. The UV light energy was monitored by the ZETA 7011-A Dosimeter-Radiometer with the energy intensity about 225 mw/cm². Additional examples (Test-2,3,4,5,6,7,8) were prepared in a manner similar to Test-1, as indicated in Table X.

Substrate (1″×2″ glass slide) was prepared by sequential application of 70% IPA (Isopropyl Alcohol) and 100% ethanol (EtOH) and then dried in air. For Ex-1A, CuxO/P25 powder was dispersed in 100% EtOH at 2 mg/mL concentration and then 100 uL of the suspension was applied to the substrate, and then dried. The application process was repeated 5 times to attain 1 mg of CuxO/P25 on the substrate. The substrate was then dried at room temperature. The coated substrates were placed in a glass dish with a water soaked filter paper for maintaining moisture, and glass spacers were inserted between the substrate and the filter paper to separate them. Ex-1B and Ex-1C were prepared in the same manner as Ex-1A. CE-1a, -1b, and -1c were prepared in the same manner as Ex-1A, except that P25 powder was used instead of CuxO/P25 powder.

### Example 3

### Photocatalytic Inactivation of S. Aureus (ATCC 6538)

S. aureus (ATCC 6538) was streaked onto a 10 cm diameter petri dish containing about 25 ml of LB agar, and was incubated at about 37° C. overnight. For each experiment, a single colony was picked to inoculate about 3 mL nutrient broth, and the inoculated culture was incubated at about 37° C. for about 16 hours to create an overnight culture (˜10̂9 cells/mL). A fresh log-phase culture was obtained by diluting the overnight culture 100×, and then incubated at about 37° C. for about 2.5 hr. The fresh culture was diluted 50×, which gave a cell suspension of about 2×10̂6 cells/mL. 50 uL of the cell suspension was pipetted onto each coated glass and/or PET substrate. A sterilized (in 70% and then 100% EtOH) plastic film (20 mm×40 mm) was placed over the suspension to spread the suspension evenly under the film. The specimen was then positioned under a fluorescent lamp, which provided ˜800 lx of light at the specimen surface. At chosen time point, e.g., after about 5 minutes, the specimen was removed from the glass dish and placed in 10 mL of 0.85% saline and vortexed at 3200 rpm for about 1 min to wash off the bacteria. The wash off suspension was serially diluted using 0.85% saline, and plated on LB agar and incubated at about 37° C. overnight to determine the number of viable cells in terms of CFU/Specimen. Counting was performed by visual inspection and the result multiplied by the dilution factor to arrive at the determined number. The results are shown in Table 2, wherein the CuₓO/P₂5 sample (EX-1b) showed at least 1 log reduction, whereas the Comparative Example 1 showed only a 0.1 to about 0.4 log reduction. See Table 2 Below.

TABLE 2

| | | 5 min, light | Log | Inoculation |
|---|---|---|---|---|
| | | S. aureus | reduction | control |
| Control | EX-1a | 1 | 2.3 | 450 |
| | EX-1b | 0 | 2.6 | 450 |
| | EX-1c | 1 | 2.3 | 450 |
| | CE-1a | 308 | 0.1 | 450 |
| | CE-1b | 176 | 0.4 | 450 |
| | CE-1c | 204 | 0.3 | 450 |
| Photocatalytic coated | Test 1 | 38 | 1.1 | 553 |
| PET film | Test 2 | 2 | 2.2 | 553 |
| | Test 3 | 0 | 2.6 | 440 |
| | Test 4 | 8 | 1.6 | 440 |
| | Test 5 | 4 | 1.9 | 440 |
| | Test 6 | 3 | 2.0 | 440 |
| | Test 7 | 1 | 2.3 | 440 |
| | Test 8 | 0 | 2.6 | 440 |

### Embodiments

The following non-limiting embodiments are contemplated.

Embodiment 1: A medical device element comprising: an acoustically transmissive element having a first side and a second side; and an antimicrobial element, the antimicrobial element disposed on or within the first side of the acoustically transmissive element; wherein the medical device element has a device first side and a device second side, and wherein the device first side contacts a patient body surface.

Embodiment 2: The medical device element of embodiment 1, wherein the acoustically transmissive element has a sound transmission with less than 5 decibels of transmission loss.

Embodiment 3: The medical device element of embodiment 1 or embodiment 2, wherein the antimicrobial element has a sound transmission with less than 0.5 decibels of transmission loss.

Embodiment 4: The medical device element of embodiment 1, embodiment 2, or embodiment 3, wherein the acoustically transmissive element comprises a substrate having a first acoustically transmissive element side and second acoustically transmissive element side, and wherein the antimicrobial element comprises a layer disposed upon the first acoustically transmissive element side.

Embodiment 5: The medical device element of embodiment 1, embodiment 2, or embodiment 3, wherein the acoustically transmissive element comprises a matrix material, and wherein the antimicrobial element is disposed within said matrix material.

Embodiment 6: The medical device element of embodiment 5, wherein the matrix material comprises a portion adjacent to a patient interface surface, wherein the antimicrobial element is disposed within the portion adjacent to the patient interface surface.

Embodiment 7: The medical device element of embodiment 1, embodiment 2, embodiment 3, embodiment 4, embodiment 5, or embodiment 6, wherein the medical device element is an externally contacting device configured to contact at least a portion of a patient's body.

Embodiment 8: The medical device element of embodiment 7, wherein externally contacting device is selected from one of the following: a stethoscope diaphragm, an echocardiogram device, an acoustic probe based on the Doppler effect, an ultrasound wand, a transducers, and a probes.

Embodiment 9: A method for manufacturing a medical device element of embodiment 1, embodiment 2, embodiment 3, embodiment 4, embodiment 5, embodiment 6, or embodiment 7, comprising combining the acoustically transmissive element with the antimicrobial element.

Embodiment 10: The method of embodiment 9, wherein the medical device element is formed at least in part by a sputtering process.

Embodiment 11: The method of embodiment 9, wherein the medical device element is formed at least in part by a spin-coating method.

Embodiment 12: The method of embodiment 9, wherein the medical device element is formed at least in part by a tape-casting process.

Embodiment 13: The medical device element of embodiment 1, embodiment 2, embodiment 3, embodiment 4, embodiment 5, embodiment 6, embodiment 7, or embodiment 8, wherein the element when adhered to a surface exhibits an adhesion of 4 B-5 B, as determined by ASTM-3363.

Embodiment 14: The medical device element of embodiment 1, embodiment 2, embodiment 3, embodiment 4, embodiment 5, embodiment 6, embodiment 7, or embodiment 8, wherein the element exhibits at least a log 1 decrease in the bacterial population after 30 minutes exposure to the antibacterial element.

Embodiment 15: The medical device element of any of embodiment 1, embodiment 2, embodiment 3, embodiment 4, embodiment 5, embodiment 6, embodiment 7, or embodiment 8, wherein the element exhibits at least a log 1 decrease in the bacterial population after 5 minutes exposure to the antibacterial element.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A medical device element comprising:
an acoustically transmissive element having a first side and a second side; and
an antimicrobial element having a first side and a second side, the antimicrobial element disposed on or within the first side of the acoustically transmissive element;
wherein the medical device element has a device first side and a device second side, and
wherein the device first side comprises at least a portion of the antimicrobial element and contacts a patient body surface.

2. The medical device element of claim 1, wherein the acoustically transmissive element has a sound transmission with less than 5 decibels of transmission loss.

3. The medical device element of claim 1, wherein the antimicrobial element has a sound transmission with less than 0.5 decibels of transmission loss.

4. The medical device element of claim 1, wherein the acoustically transmissive element comprises a substrate having a first acoustically transmissive element side and second acoustically transmissive element side, and wherein the antimicrobial element comprises a layer disposed upon the first acoustically transmissive element side.

5. The medical device element of claim 1, wherein the acoustically transmissive element comprises a matrix material, and wherein the antimicrobial element is disposed within said matrix material.

6. The medical device element of claim 5, wherein the matrix material comprises a portion adjacent to a patient interface surface, wherein the antimicrobial element is disposed within the portion adjacent to the patient interface surface.

7. The medical device element of claim 1, wherein the medical device element is an externally contacting device configured to contact at least a portion of a patient's body.

8. The medical device element of claim 7, wherein externally contacting device is selected from one of the following: a stethoscope diaphragm, an echocardiogram device, an acoustic probe based on the Doppler effect, an ultrasound wand, a transducers, and a probes.

9. A method for manufacturing a medical device element of claim 1, comprising combining the acoustically transmissive element with the antimicrobial element.

10. The method of claim 9, wherein the medical device element is formed at least in part by a sputtering process.

11. The method of claim 9, wherein the medical device element is formed at least in part by a spin-coating method.

12. The method of claim 9, wherein the medical device element is formed at least in part by a tape-casting process.

13. The medical device element of claim 1, wherein the element when adhered to a surface exhibits an adhesion of 4 B-5 B.

14. The medical device element of claim 1, wherein the element exhibits at least a log 1 decrease in the bacterial population after 30 minutes exposure to the antibacterial element.

15. The medical device element of any preceding claim, wherein the element exhibits at least a log 1 decrease in the bacterial population after 5 minutes exposure to the antibacterial element.
